(19) United States
(12) Patent Application Publication
(10) Pub. No.: US 2026/0207580 A1
(43) Pub. Date: Jul. 23, 2026

(54) NITROXOLINE FOR USE IN THE TREATMENT OR PREVENTION OF A MALIGNANT PERIPHERAL NERVE SHEATH TUMOUR

(71) Applicant: HEALX LTD, Cambridge (GB)

(72) Inventor: David BROWN, Cambridge (GB)

(21) Appl. No.: 19/136,354

(22) PCT Filed: Dec. 8, 2023
(86) PCT No.: PCT/GB2023/053167

§ 371 Date: Jun. 6, 2025

(30) Foreign Application Priority Data
Dec. 8, 2022 (GB) 2218460.0

(51) Int. Cl. A61K 31/47; A61P 35/00

(52) U.S. Cl. CPC A61K 31/47; A61P 35/00

(57) ABSTRACT

The present invention relates to a composition comprising nitroxoline, or a pharmaceutically acceptable salt thereof, for use in the treatment or prevention of a MPNST.

### FIELD OF THE INVENTION

This invention relates to new uses of nitroxoline.

### BACKGROUND OF THE INVENTION

Nitroxoline is used in humans as an antibiotic, it is not widely used but has been on the market since the 60s. It is used in the treatment or prevention of biofilm infections, such as urinary tract infections. It is particularly effective at disrupting biofilms and it is the metal cation chelation property that is believed to be responsible for this action. Nitroxoline is metabolised in the liver to the corresponding sulphate and glucuronide metabolites. There is evidence that the metabolites both share the antimicrobial activity. It has also been used in anticancer settings via antiproliferative action. Nitroxoline has the systematic name 5-nitroquinolin-8-ol.

A malignant peripheral nerve sheath tumour (MPNST) is a form of cancer of the connective tissue surrounding nerves. A sarcoma is defined as a MPNST when at least one of the following criteria is met: it arises from a peripheral nerve, it arises from a pre-existing benign nerve sheath tumor (neurofibroma) or it demonstrates Schwann cell differentiation on histologic examination. MPNSTs are considered aggressive and are associated with a low survival rate.

MPNSTs usually present as an enlarging palpable mass. Pain is a variable complaint. Rapid enlargement occurs more often in the setting of NF1 and should raise concern for malignant degeneration of a neurofibroma. MPNSTs arising from peripheral nerves may result in a variety of clinical patterns, including radicular pain, paresthesias, and motor weakness.

Many MPNSTs arise from plexiform neurofibromas (benign nerve-sheath tumours), which themselves arise during early development from nerves in the skin, or from more internal nerve bundles such as cranial nerves or proximal large peripheral nerve sheaths, Plexiform neurofibromas have a 10-15% lifetime incidence of transformation to a MPNST.

Many people with a MPNST are also found to have neurofibromatosis type I (NF1), an autosomal-dominant genetically inherited disease. NF1 is caused by germline mutations of the NF1 tumor suppressor gene, which encodes the protein neurofibromin. Neurofibromin functions as a GTPase-activating (GAP) protein and inactivates the intracellular signal transduction protein Ras by converting the active GTP-bound form into its inactive GDP-bound form. This in turn leads to the downregulation of Ras activity. Loss of neurofibromin activity increases Ras activity, which in turn promotes the transcription of a number of genes required for cell growth and proliferation.

Longitudinal imaging studies of patients with plexiform neurofibromas have identified a subset of distinct nodular lesions that emerge within existing plexiform neurofibromas, grow rapidly relative to the surrounding tumor, and are FDG-PET avid [Evans, D. G., et al., J Med Genet, 2002. 39(5): p. 311-4.]. Biopsies often reveal atypical neurofibromatous neoplasms of uncertain biologic potential (ANNUBP), which share overlapping histopathological features with MPNSTs and have been associated as potential MPNST precursors [Miettinen, M. M., et al., Hum Pathol, 2017. 67: p. 1-10]. 9p21.3 deletions encoding the entire CDKN2A/B locus (INK4/ARF locus), have been identified as a solitary and highly recurrent genetic aberration in the majority of human ANNUBP (94%, n=15/16). Further, haplo-insufficient or homozygous loss of CDKN2A (p16^{INK4A}) and its alternative reading frame p14^{ARF} was identified in 60-80% of MPNSTs in two recent independent studies [Brohl, A. S., et al., Sci Rep, 2017. 7(1): p. 14992; Lee, W., et al., Nat Genet, 2014. 46(11): p. 1227-32].

The Ink4a/Arf tumor suppressor is pivotal to restraining plexiform neurofibroma progression by inducing a broad signature of senescence. Mice harboring conditional ablation of Nf1 and Ink4a/Arf in embryonic neural crest spontaneously develop tumors that are histopathologically indistinguishable from human ANNUBP and subsequently progress to a MPNST with high penetrance [Rhodes, S. D., et al., Hum Mol Genet, 2019]. This tissue lineage-specific model is the first to recapitulate the malignant transformation of pre-existing plexiform neurofibroma and ANNBUP precursor lesions as seen in human patients.

Current MPNST therapies, including surgery and chemoradiotherapy, are both debilitating and largely ineffective. After 10 years, the overall survival rate is only around 50% for patients with NF-1 associated MPNST. This has highlighted the need for new therapies.

### SUMMARY OF THE INVENTION

The present inventors have found that nitroxoline inhibits cell proliferation and increases apoptosis in vitro in stem cells that recapitulate transformed Nf1 MPNST. It is thus expected that nitroxoline will reduce, treat and prevent a MPNST.

Accordingly, the present invention is a composition comprising nitroxoline, or a pharmaceutically acceptable salt thereof, for use in the treatment or prevention of a MPNST.

A first aspect of the invention is a composition comprising nitroxoline, or a pharmaceutically acceptable salt thereof, for use in the treatment or prevention of a MPNST.

A second aspect of the invention is use of nitroxoline, or a pharmaceutically acceptable salt thereof, for the manufacture of a medicament for use in the treatment or prevention of a MPNST.

A third aspect of the invention provides a method of treating or preventing a MPNST comprising administering the patient with a composition comprising nitroxoline or a pharmaceutically acceptable salt thereof.

### DESCRIPTION OF THE FIGURES

FIG. 1 shows the nitroxoline dose-response in proliferation and apoptosis of Nf1^{-/-} and Nf1^{-/-}; Ink4a/Arf^{+/-} DNSCs in in vitro assays.

### DETAILED DESCRIPTION

In the present invention, and as demonstrated by the data herein, nitroxoline inhibits cell proliferation and increases apoptosis in vitro in stem cells that recapitulate transformed Nf1 MPNST, and is therefore an effective treatment of a MPNST. Preferably, nitroxoline is used for the treatment or prevention of a MPNST, wherein the subject has neurofibromatosis type I.

By the term “treatment” or “treating” as used herein, we refer to therapeutic (curative) treatment, which includes reducing the size of a MPNST. A biopsy may be used to diagnose MPNSTs. By the term “prevention” or “preventing” as used herein, we refer to “prophylactic” treatment, which includes administering nitroxoline to a patient to avoid a MPNST developing, for example a patient that has a plexiform neurofibroma but a MPNST has not developed. The plexiform neurofibroma may have begun to proliferate, such as to proliferate rapidly,

“Patient” and “subject” are used interchangeably and refer to the subject that is to be administered the nitroxoline. Preferably the subject is a human. Suitably the subject has neurofibromatosis, preferably neurofibromatosis type I.

In one embodiment, nitroxoline is used for the treatment or prevention of a MPNST, wherein the patient has had or is going to have surgery to remove some or all of the MPNST. This may be particularly advantageous if the MPNST is large and/or expands across tissue boundaries, so it is difficult to remove it all by surgery and/or a quick removal of at least some of it is desired/beneficial.

In one embodiment, nitroxoline is used for the prevention of a MPNST, wherein the patient has had or is going to have surgery to remove some or all of a plexiform neurofibroma. Typically in this embodiment, the plexiform neurofibroma has not developed into a MNPST.

The term “surgery” has its normal meaning in the art. Surgery is an invasive technique with the fundamental principle of physical intervention on organs/organ systems/tissues for diagnostic or therapeutic reasons.

In one aspect, the patient has a genetic aberration such as 9p21.3 deletions encoding the entire CDKN2A/B locus (INK4/ARF locus), or, haplo-insufficient or homozygous loss of CDKN2A (p16^{INK4A}) and its alternative reading frame p14^{ARF}. Such genetic aberrations have been identified as a solitary and highly recurrent in the majority of human ANNUBP associated with MPNSTs.

As used herein, a pharmaceutically acceptable salt is a salt with a pharmaceutically acceptable acid or base. Pharmaceutically acceptable acids include both inorganic acids such as hydrochloric, sulphuric, phosphoric, diphosphoric, hydrobromic or nitric acid and organic acids such as citric, fumaric, maleic, malic, ascorbic, succinic, tartaric, benzoic, acetic, methanesulfonic, ethanesulfonic, salicylic, stearic, benzenesulfonic or p-toluenesulfonic acid. Pharmaceutically acceptable bases include alkali metal (e.g. sodium or potassium) and alkali earth metal (e.g. calcium or magnesium) hydroxides and organic bases such as alkyl amines, aryl amines or heterocyclic amines.

The present invention is directed to a composition comprising nitroxoline, or a pharmaceutically acceptable salt thereof, for use in the treatment or prevention of a MPNST.

In an alternative embodiment, the present invention is directed to a composition comprising nitroxoline, or a pharmaceutically acceptable salt thereof, for use in the treatment or prevention of a MPNST, wherein nitroxoline is the only active agent in the composition. By only active agent it is meant that the composition does not contain other components which may be used in the treatment or prevention of a MPNST.

Alternatively, the composition comprising nitroxoline, or a pharmaceutically acceptable salt thereof, may also comprise one or more active agents in the composition, preferably one. The additional active agent(s) may be an agent active for treating or preventing MPNST.

The compositions of the invention may contain a pharmaceutically acceptable carrier. By “pharmaceutically acceptable carrier” is meant any diluent or excipient, such as fillers or binders, that is compatible with the other ingredients of the composition, and which is not deleterious to the recipient. The pharmaceutically acceptable carrier can be selected on the basis of the desired route of administration, in accordance with standard pharmaceutical practices.

In the present invention, the composition may be administered in a variety of dosage forms. In one embodiment, the composition may be formulated in a format suitable for oral, rectal, parenteral, intranasal or transdermal administration or administration by inhalation or by suppository.

The composition may be administered orally, for example as tablets, troches, lozenges, aqueous or oily suspensions, dispersible powders or granules. Preferably, the composition is formulated such that it is suitable for oral administration, for example tablets and capsules. Tablets and capsules may be prepared with binding agents, for example, syrup, acacia, gelatin, sorbitol, tragacanth, celluloses or polyvinylpyrrolidone; fillers, such as lactose, sucrose, corn starch, calcium phosphate, sorbitol, or glycine; lubricants, such as magnesium stearate, talc, polyethylene glycol, or silica; and surfactants, such as sodium lauryl sulfate. Liquid compositions may contain conventional additives such as suspending agents, for example sorbitol syrup, methyl cellulose, sugar syrup, gelatin, carboxymethyl-cellulose, or edible fats; emulsifying agents and surfactants such as lecithin, or acacia; vegetable oils such as almond oil, coconut oil, cod liver oil, or peanut oil; preservatives such as butylated hydroxyanisole (BHA) and butylated hydroxytoluene (BHT). Liquid compositions may be encapsulated in, for example, gelatin to provide a unit dosage form.

The composition may also be administered parenterally, whether subcutaneously, intravenously, intramuscularly, intrasternally, transdermally or by infusion techniques.

The composition may also be administered by inhalation. An advantage of inhaled medications is their direct delivery to the area of rich blood supply in comparison to many medications taken by oral route. Thus, the absorption is very rapid as the alveoli have an enormous surface area and rich blood supply and first pass metabolism is bypassed.

The present invention also provides an inhalation device containing the composition of the present invention. Typically said device is a metered dose inhaler (MDI), which contains a pharmaceutically acceptable chemical propellant to push the medication out of the inhaler.

The composition may also be administered by intranasal administration. The nasal cavity's highly permeable tissue is very receptive to medication and absorbs it quickly and efficiently. Nasal drug delivery is less painful and invasive than injections, generating less anxiety among patients. By this method absorption is very rapid and first pass metabolism is usually bypassed, thus reducing inter-patient variability. Further, the present invention also provides an intranasal device containing the composition according to the present invention.

The composition may also be administered by transdermal administration. For topical delivery, transdermal and transmucosal patches, creams, ointments, jellies, solutions or suspensions may be employed. The present invention therefore also provides a transdermal patch containing the composition.

The composition may also be administered by sublingual administration. The present Invention therefore also provides a sub-lingual tablet comprising the composition.

The composition may also be formulated with an agent which reduces degradation of the substance by processes other than the normal metabolism of the patient, such as anti-bacterial agents, or inhibitors of protease enzymes which might be the present in the patient or in commensural or parasite organisms living on or within the patient, and which are capable of degrading the compound.

Liquid dispersions for oral administration may be syrups, emulsions and suspensions.

Suspensions and emulsions may contain as carrier, for example a natural gum, agar, sodium alginate, pectin, methylcellulose, carboxymethylcellulose, or polyvinyl alcohol. The suspension or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for injection or infusion may contain as carrier, for example, sterile water or preferably they may be in the form of sterile, aqueous, isotonic saline solutions.

In an embodiment of the invention, the composition is administered in an effective amount to treat or prevent a MPNST. An effective dose will be apparent to one skilled in the art, and is dependent on a number of factors including age, sex, weigh, which the medical practitioner will be capable of determining.

In a preferred embodiment, the composition comprises 30 mg to 600 mg, preferably 50 mg to 500 mg, more preferably 100 mg to 400 mg, yet more preferably 150 mg to 350 mg, most preferably 200 mg to 300 mg nitroxoline.

The composition may be administered once a day, twice a day, three times a day or four times a day.

In an embodiment of the invention, the composition is administered at least once a day. Preferably it is administered as a single daily dose. Preferably the single daily dose is 90 mg to 1800 mg, preferably 150 mg to 1500 mg, more preferably 300 mg to 1200 mg, yet more preferably 450 mg to 1050 mg, most preferably 600 mg to 900 mg of nitroxoline.

In an embodiment of the invention, the composition is administered twice daily. Preferably each dose is 45 mg to 900 mg, preferably 75 mg to 750 mg, more preferably 150 mg to 600 mg, yet more preferably 225 mg to 525 mg, most preferably 300 mg to 450 mg of nitroxoline.

In an embodiment of the invention, the composition is administered three times daily. Preferably each dose is 30 mg to 600 mg, preferably 50 mg to 500 mg, more preferably 100 mg to 400 mg, yet more preferably 150 mg to 350 mg, most preferably 200 mg to 300 mg of nitroxoline.

In an embodiment of the invention, the composition is administered four times daily. Preferably each dose is 15 mg to 500 mg, preferably 50 mg to 400 mg, more preferably 100 mg to 300 mg, yet more preferably 125 mg to 225 mg, most preferably 150 mg to 200 mg of nitroxoline.

Preferably, the dosage regime is such that the total daily dosage of nitroxoline does not exceed 1500 mg.

Suitably the effective dose of nitroxoline results in a concentration of 1 to 150 μM, preferably 10 to 100 μM, more preferably 25 to 50 μM in cells.

In order to treat or prevent a MPNST, the composition comprising nitroxoline is used in a chronic dosage regime i.e. chronic, long-term treatment. Suitably the regime lasts for at least one month, suitably at least two months, such as at least three months.

The present invention also relates to use of nitroxoline, or a pharmaceutically acceptable salt thereof, for the manufacture of a medicament for use in the treatment or prevention of a MPNST. This embodiment of the invention may have any of the preferred features described above.

The present invention also relates to a method of treating or preventing a MPNST comprising administering the patient with a composition comprising nitroxoline or a pharmaceutically acceptable salt thereof. This embodiment of the invention may have any of the preferred features described above. The method of administration may be according to any of the routes described above.

For the avoidance of doubt, the present invention also embraces prodrugs which react in vivo to give a compound of the present invention.

### Experimental Section

### In Vitro Drug Screen Utilizing Nf1 and Nf1-Ink4a/Arf Mutant DNSCs

Nf1-Ink4a/Arf deficient cells contained with the embryonic dorsal root ganglia (DRG)/nerve roots (DNSCs) can be re-implanted into the nerve microenvironment and give rise to tumors histologically indistinguishable from human ANNBUP within 6 weeks. These lesions further progress to high grade MPNST with 100% penetrance by 3-4 months post implantation. By providing a high efficiency of tumorigenesis in a defined latency period, this approach will provide a robust platform for evaluation of experimental therapeutics designed to treat, delay, and or prevent progression of ANNUBP and a MPNST driven by loss of Nf1 and Ink4/Arf [Chen, Z., et al., Cancer Cell, 2014. 26(5): p. 695-706].

Nf1-Ink4a/Arf mutant DNSCs have increased cell cycle activity and proliferation in vitro, consistent with the in vivo phenotypes. The present study utilizes Nf1^{-/-} and Nf1^{-/-}; Ink4a/Arf^{+/-} mutant DNSCs obtained by transient infection of Nf1^{flox/flox} and Nf1^{flox/flox}; Ink4a/Arf^{flox/+} DNSCs with an adenovirus carrying Cre recombinase. Selectivity of nitroxilone was compared between Nf1^{-/-} and Nf1^{-/-}; Ink4a/Arf^{+/-} DNSCs.

Nf1^{-/-} and Nf1^{-/-}; Ink4a/Arf^{+/-} DNSCs described above were treated with serially diluted nitroxoline starting from 105 μM and incubated for 48 hrs. After the incubation period, proliferation, viability and apoptosis assays were performed as described below:
DNSC proliferation and viability was assessed using the CellTiter-Glo Assay (Promega) which measures ATP consumption. Briefly, Nf1^{-/-} and Nf1^{-/-}; Ink4a/Arf^{+/-} DNSCs were plated in triplicate at a concentration of 5,000 cells/well in 96-well dishes in 100 μl DNSC growth medium supplemented with FGF and EGF and placed in a 37° C., 5% CO₂ humidified incubator with or without the addition of the experimental compounds for 48 hours. Following incubation, 100 μL of CellTiterGlo reagent was added to each well. After 10 minutes, the plates were read using a 96-well microplate luminometer.
To assess cellular apoptosis, the Caspase-Glo 3/7 kit (Promega) was utilized according to the manufacturer's instructions. Nf1^{-/-} and Nf1^{-/-}; Ink4a/Arf^{+/-} DNSCs were plated and treated with experimental compound. After 48 hours, 100 μL Caspase-Glo 3/7 reagent was added to each well. After 10 minutes, plates were read using a 96 well luminometer to measure caspase 3/7 activity.

### Results

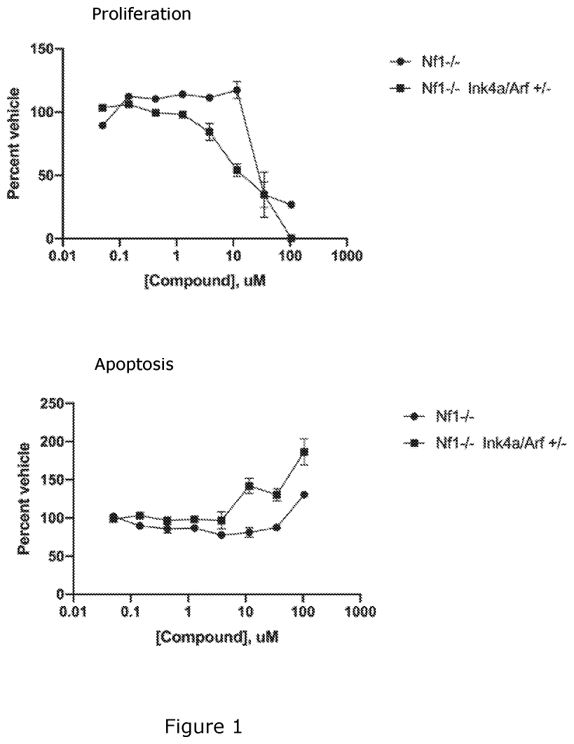

Nitroxoline inhibited cell proliferation and increased apoptosis in a dose response manner, as seen in FIG. 1. The antiproliferative effect was observed at concentrations greater than 3 μM in the cells Nf1−/−Ink4a/Arf−/− and at concentrations greater than 10 μM in the cells Nf1−/−. The induction of apoptosis was evident at concentrations greater than 10 μM in the cells Nf1−/−Ink4a/Arf−/− and at concentrations between 30-100 μM in the nf1−/− cells.

### Conclusions

Nitroxoline inhibits cell proliferation and increases apoptosis in vitro in stem cells that recapitulate transformed Nf1MPNST. It is thus expected that nitroxoline will reduce, treat and prevent a MPNST.

1. A method of treating or preventing a malignant peripheral nerve sheath tumour (MPNST), said method comprising administering to a subject a composition comprising nitroxoline, or a pharmaceutically acceptable salt thereof.

2. The method according to claim 1, wherein the method is a method of treating the MPNST.

3. The method according to claim 1, wherein the subject of the treatment or prevention has neurofibromatosis, preferably neurofibromatosis type I.

4. The method according to claim 1, wherein the subject of the treatment or prevention is human.

5. The method according to claim 1, wherein the composition comprises 30 mg to 600 mg of nitroxoline.

6. The method according to claim 1, wherein administration is by a dose two times per day.

7. The method according to claim 6, wherein the dose is 45 mg to 900 mg of nitroxoline.

8. The method according to claim 1, where administration is by a dose three times per day.

9. The method according to claim 8, wherein the dose is 30 mg to 600 mg of nitroxoline.

10. The method according to claim 1, where administration is by a dose four times per day.

11. The method according to claim 10, wherein the dose is 15 mg to 500 mg of nitroxoline.

12. The method according to claim 1, wherein the composition is administered orally or intravenously.

13. The method according to claim 1, wherein the composition is administered by parenteral, transdermal, sublingual, rectal or inhaled administration.

14. The method according to claim 1, wherein the nitroxoline is the only active agent in the composition.

15-18. (canceled)

19. The method according to claim 5, wherein the composition comprises 200 mg to 300 mg of nitroxoline.

20. The method of according to claim 7, wherein the dose is 300 mg to 450 mg of nitroxoline.

23. The method of according to claim 9, wherein the dose is 200 mg to 300 mg of nitroxoline.

24. The method of according to claim 11, wherein the dose is 150 mg to 200 mg of nitroxoline.
